(19) United States
(12) Patent Application Publication
(10) Pub. No.: US 2026/0345259 A1
(43) Pub. Date: Nov. 5, 2026

(54) Perioperative Cobotic System with Modular End Effectors

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,144

(22) Filed: Dec. 24, 2025

Related U.S. Application Data
(63) Continuation-in-part of application No. 19/195,731, filed on Apr. 30, 2025.

(51) Int. Cl. A61B 34/30; B25J 9/16; B25J 15/00; B25J 15/04; B25J 19/06

(52) U.S. Cl. CPC A61B 34/30; B25J 9/162; B25J 15/0019; B25J 15/0483; B25J 19/06

(57) ABSTRACT

A mobile perioperative cobotic system is disclosed for performing support tasks in surgical and sterile processing environments using modular, interchangeable end effector tools. The system includes a mobile robotic platform with an articulated manipulator, a standardized mechanical and electrical interface for rapid tool attachment and detachment, and a library of task-specific end effectors including grippers, suction and irrigation nozzles, suture tools, sensor probes, and sterilization modules. A recognition and configuration subsystem automatically detects attached tools and adapts control parameters accordingly. A predictive tool selection engine anticipates upcoming task needs based on procedural context and interfaces with scheduling systems to preload appropriate tools. The system maintains sterile field compliance through automated sterility checks, proximity sensing, and barrier integration. User interaction is provided via voice or visual interface, with override and emergency disengagement controls. All tool interactions are logged for traceability, sterilization tracking, and predictive maintenance.

### CROSS-REFERENCE TO RELATED APPLICATIONS

This patent application is a continuation-in-part of U.S. patent application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present invention relates generally to robotic systems in surgical environments, and more particularly to cobotic platforms equipped with modular, task-adaptive end effector systems for performing perioperative support functions including surgical assistance, sterile logistics, and instrument handling.

### BACKGROUND

Modern operating rooms are increasingly reliant on robotic assistance to enhance precision, reduce manual burden, and optimize surgical workflows. Collaborative robots, or “cobots,” are particularly well-suited to perioperative environments due to their ability to safely interact with human personnel while executing repetitive or support-intensive tasks. However, existing cobotic systems are typically designed for fixed functionalities and are constrained by limited adaptability to the wide range of intraoperative and perioperative tasks that arise across different surgical procedures.

Many current cobots lack the ability to rapidly reconfigure their tooling to accommodate different support roles, such as instrument transport, suction, drape positioning, suture handling, or localized sterilization. When task requirements change, the transition often requires manual tool changes or the involvement of multiple dedicated robotic units—interrupting workflow and creating sterility risks. Moreover, these systems generally do not include intelligent recognition or predictive logic to automate tool selection or enforce context-aware restrictions based on surgical phase, clinical authorization, or sterility status.

Additionally, most existing tool-mounted systems lack a standardized interface for end effector interchange, resulting in slow reconfiguration, poor interoperability, and elevated risk of operator error. Conventional robots also rely on user-initiated commands for tool changes, with little or no automation in anticipating procedural needs, adapting movement profiles, or tracking tool-specific usage and sterilization history. Such limitations reduce the flexibility and responsiveness of robotic systems in complex surgical settings.

There is therefore a recognized need for a cobotic platform that supports modular, interchangeable end effector tooling with rapid, sterile-compatible coupling and uncoupling; automated tool recognition and configuration; task-and workflow-aware preloading; and real-time sterility compliance enforcement. Such a system would increase the functional versatility of perioperative cobots while improving procedural efficiency, reducing staff burden, and ensuring safety and traceability in the surgical environment.

### SUMMARY

The present invention provides a perioperative cobotic system configured with a modular end effector interface to enable dynamic, task-specific tool interchange in sterile surgical environments. The system comprises a mobile robotic platform with a multi-degree-of-freedom manipulator arm, a standardized mechanical and electrical coupling interface for end effector tools, and a predictive tool selection engine that anticipates surgical needs based on workflow context and phase data.

The modular end effector interface supports rapid, secure attachment and detachment of a diverse set of task-specific end effectors—including dexterous hands, instrument grippers, suction and irrigation modules, drills, saws, cautery, drape manipulators, suture assistance tools, sensor probes (e.g., thermal, ultrasound, RFID), and cleaning or sterilization units. The interface maintains sterile field integrity through automatic coupling mechanisms and sterility-compliant barrier integration.

A recognition and configuration subsystem automatically detects the identity of an attached tool and configures motion profiles, control logic, and functional constraints accordingly. The system enforces compliance with sterilization protocols by verifying tool status and halting operations in response to sterility breaches. A data logging module records usage, sterilization history, and wear patterns for each end effector and interfaces with hospital inventory and asset management systems to support predictive maintenance and real-time availability tracking.

The system further includes a voice-and visually-activated user interface that allows clinicians to request, confirm, or override tool changes and includes emergency disengagement options for safety. Autonomous or semi-autonomous operation enables the cobot to retrieve, preload, and present tools in anticipation of procedural requirements, enhancing surgical team coordination and minimizing workflow interruptions.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a perioperative cobotic system.

FIG. 2 is an exploded schematic view of a modular end effector interface.

FIG. 3 is a system-level block diagram illustrating a control architecture of the perioperative cobotic system.

FIG. 4 is a flowchart illustrating a predictive end effector selection and automated tool-change workflow.

FIG. 5 is a schematic view of a user interaction interface.

FIG. 6 is a flowchart illustrating an end effector sterility verification and safety compliance protocol.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

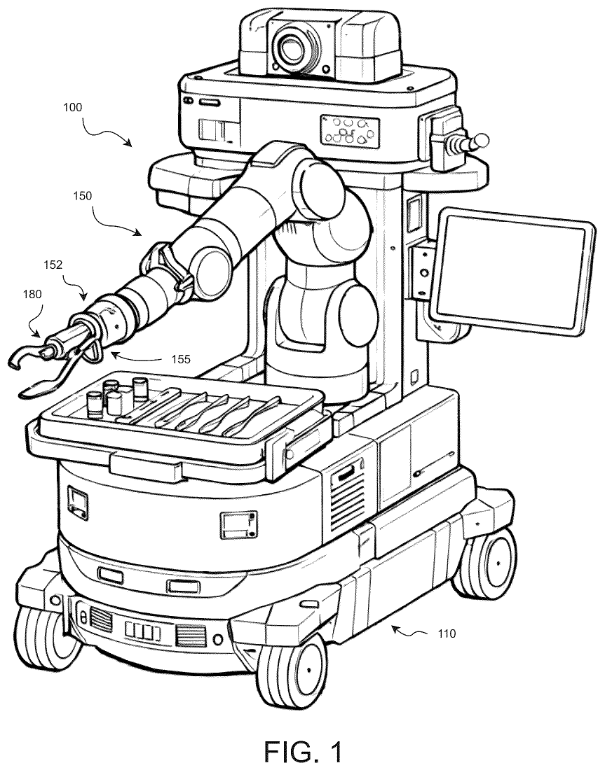

Referring to FIG. 1, a perioperative cobotic system 100 (also referred to herein as the “cobot 100”) is illustrated in a mobile configuration. The cobot 100 includes a mobile base 110 and an articulated robotic arm 150 mounted on the base. The mobile base 110 provides a stable platform and houses drive motors and wheels that enable the cobot to navigate within a crowded operating room (OR). In the depicted embodiment, the mobile base 110 is compact with a low center of gravity and may utilize a differential drive or omnidirectional wheels, allowing high maneuverability in constrained OR environments. The base 110 also contains power sources (e.g., battery packs) and onboard sensors for navigation and obstacle avoidance, permitting the cobot 100 to move around equipment and personnel while respecting sterile field boundaries. In some embodiments, the base 110 includes stabilizers or a locking mechanism to fix its position during delicate tasks, ensuring the arm's movements do not cause unintended motion. This mobility and stability combination enables the cobot 100 to be positioned optimally for various perioperative support tasks, even in dynamic and space-limited surgical suites.

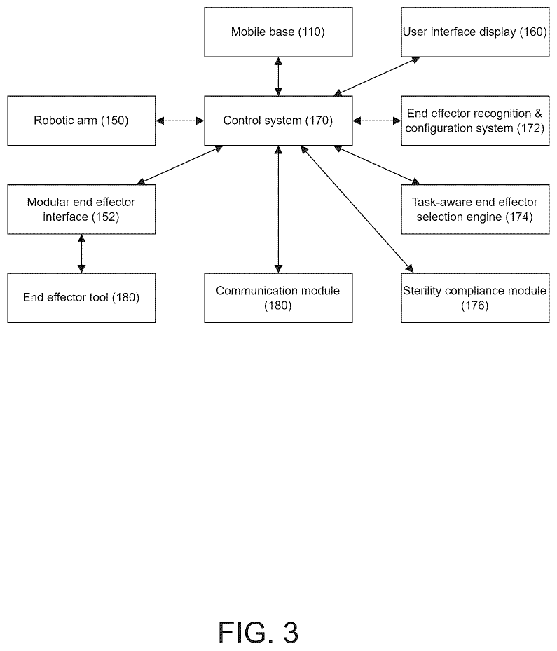

The robotic arm 150 is coupled to the mobile base and provides multiple degrees of freedom for precise positioning of end effectors. In the example of FIG. 1, the arm 150 has several articulating joints (e.g., shoulder, elbow, wrist joints) that collectively allow the end effector to reach different heights and angles around a patient or operating table. High-precision movement control is implemented so that the arm 150 can perform fine motions required for surgical assistance tasks without jitter or overshoot. The arm 150 may be a multi-axis manipulator similar to an industrial or surgical robot arm, capable of carrying various tools and applying controlled forces. A control system 170 (see FIG. 3) coordinates the mobile base 110 and arm 150 motions to avoid collisions and maintain smooth operation. By combining a movable base with an articulated arm, the cobot 100 can effectively extend its reach across the OR, for example, rolling up to the bedside and then using the arm 150 to deliver an instrument or hold a tool at the surgical site. The mobile cobot embodiment is designed to safely operate alongside the surgical team: it includes proximity sensors, 3D cameras, or LiDAR to detect obstacles and personnel, and will adjust its path or stop if a person or object is too close. This allows the cobot 100 to maneuver in real time around shifting obstacles (such as a nurse stepping into its path) while maintaining a respectful distance from the sterile field. The overall system is thus well-suited to the dynamic, congested environment of an OR, offering the flexibility to reposition as needed and perform tasks in different locations around the room.

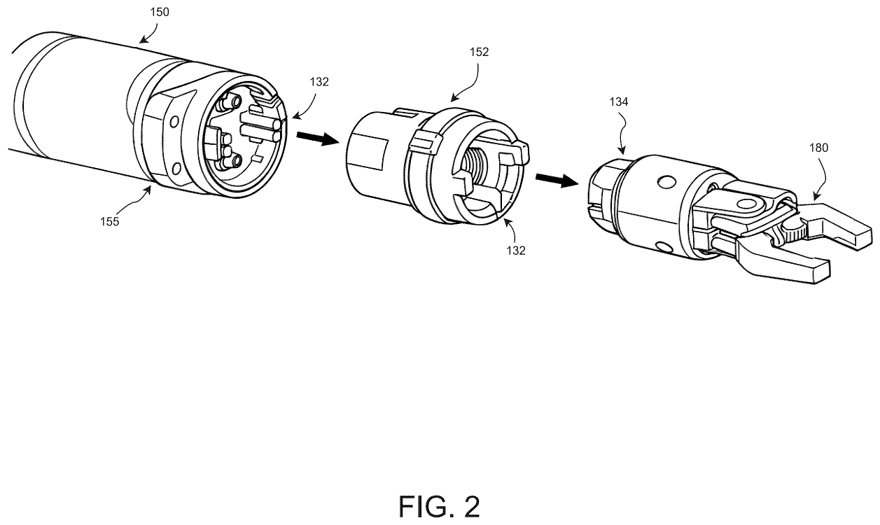

FIG. 2 illustrates an exploded schematic of the modular end effector interface 152 at the distal end 155 of the robotic arm 150. This interface 152 provides a standardized mechanical and electrical coupling for the attachment of various surgical tools (end effectors) to the arm. In the depicted embodiment, the interface 152 comprises a mating connector system with complementary components on the arm side and the tool side. For example, the arm 150 may terminate in a quick-connect hub or socket, and each end effector 180 may include a corresponding plug or adapter. As shown schematically in FIG. 2, alignment features (such as keys and keyways or guide pins 132) ensure that the tool mounts in the correct orientation and that electrical contacts properly align when the tool is attached. A locking mechanism 134 is provided to securely lock the end effector in place during use. The locking mechanism 134 may be spring-loaded or motorized, for instance, in one embodiment the arm 150 includes a solenoid-driven latch that engages a catch on the tool, or a twist-lock collar that the robot can rotate to clamp the tool. This mechanism allows rapid attachment and detachment of tools, enabling the cobot 100 to swap end effectors in a matter of seconds.

The interface 152 also incorporates electrical connectors 132 (or wireless power/data transfer coils in some embodiments) that automatically couple with matching connectors on the end effector. These connectors provide power and signal communication between the control system 170 and the tool. For example, a multi-pin electrical interface can carry power, data bus signals, and control lines so that tools with sensors or actuators are fully integrated into the cobot's control loop. In one embodiment, the electrical connection is made via spring-loaded pogo pins and pads that self-align when the tool is inserted, while another embodiment may use a small docking port with a high-speed data plug (such as USB or a custom bus) for reliable communication. The mechanical and electrical elements of interface 152 are preferably universal across the end effector library, i.e., every authorized tool is built with the same standardized coupling geometry and pinout, allowing any tool to be attached to any cobot arm interchangeably. This standardization simplifies the design and ensures that adding new tools in the future does not require hardware changes to the arm.

Maintaining sterility during tool swaps is a critical aspect of the interface design. Accordingly, the modular interface 152 is designed with sterile barrier integration and minimal exposed moving parts. In some embodiments, a sterile drape or sleeve covers the distal portion of the arm 150 and interface 152 during surgery. The sleeve provides a sterile field isolation, such that only the sterile side of the adapter (in contact with the tool) is exposed, while the arm's non-sterile surfaces remain covered beneath the drape. The end effectors themselves are sterilized (e.g., via autoclave or gas sterilization) prior to the procedure and may be kept in a sterile container or rack until the cobot retrieves them. The interface 152's mechanical design can accommodate this by, for example, using a disposable sterile coupling insert, a removable sterile adapter that snaps onto the arm's coupling. In use, the cobot 100 would first be fitted with the sterile adapter (ensuring the coupling surfaces are all sterile), and then tools can be attached to the adapter without contaminating them. FIG. 2 shows an example exploded view where the end effector 180 aligns with the arm's distal end 155 via such an adapter and locking collar 134. The electrical coupling is enclosed in a sealed housing with gaskets to prevent ingress of fluids or contaminants, allowing it to be easily cleaned and sterilized between uses. Furthermore, because the interface supports automatic or semi-automatic tool changes without manual intervention, the system can perform a swap while preserving sterile conditions by moving to a designated swap location away from the patient (if needed) and using only the pre-sterilized components during the exchange. In an embodiment, the cobot 100 may even include an integrated sterilization feature at the interface, for instance, a UV light emitter or alcohol spray nozzle around the coupling, to quickly disinfect the coupling surfaces before attaching a new tool. This mechanical and electrical interface design thus enables modularity of the cobot's functionality, allowing a wide variety of end effectors to be attached reliably, safely, and with minimal downtime or contamination risk.

Using the modular interface 152, the cobot 100 supports a suite of task-specific interchangeable end effectors. These end effectors 180 (examples of which are illustrated conceptually in FIG. 1 and FIG. 2 attached to arm 150) transform the cobot into different tools depending on the surgical task at hand. The following are representative examples of the end effector tools in the system's library:

Grippers: Mechanical gripper hands or clamps capable of grasping and carrying objects. A gripper end effector may be used for instrument transport, for example, fetching a surgical instrument or an implant from a tray and delivering it to the surgeon, or disposing of used items. The grippers are designed to handle delicate instruments without damage and may include force sensors to regulate grip pressure and/or Hall effect sensors to improve adherence to certain instruments.

Suction/Irrigation Nozzles: A combined suction and irrigation tool that can connect to hospital suction lines or fluid reservoirs. This tool allows the cobot to assist with suctioning blood or fluids from the surgical site and irrigating wounds as directed. The nozzle end effector typically includes tubing connectors (e.g., a quick-connect for suction tubing) and valves that the cobot's control system can open/close to regulate flow. By attaching the suction/irrigation nozzle, the cobot essentially becomes an automated suction device that can be positioned at precise locations, freeing an assistant's hands.

Sterile Drape Holders: A specialized effector designed to hold and position surgical drapes. It may consist of an adjustable clamp or frame that can grasp the edge of a sterile drape. The cobot can use this to help drape the patient or equipment, for instance, lifting and suspending a drape while the surgical team arranges it, or holding a drape to form a barrier without needing a human to hold it. This tool aids in maintaining the sterile field by providing additional “hands” for draping tasks.

Suture Assistance Tools: These can include needle holders or devices to assist in suturing and knot tying. For example, an end effector could be a suturing tool that holds a suture needle (similar to a robotic surgical needle driver) allowing the cobot to present the needle to the surgeon or even help drive the needle through tissue under the surgeon's guidance. Another variant is a tool that holds tension on suture threads or cuts sutures when commanded. Such tools can offload simple repetitive tasks during wound closure. In other embodiments tools can include retractors, saws, drills, cautery.

Sensor Probes: Various sensing modules can be attached as end effectors to collect data or perform measurements. Examples include a thermal camera or thermometer probe, an ultrasound probe for scanning tissue, RFID or barcode scanners for reading instrument tags, camera systems, or other diagnostic sensors. When attached, the cobot can precisely position these probes, for instance, scanning a surgical site for residual sponges (with RFID) or measuring patient surface temperature at a location. The sensors feed data back through the interface 152 to the control system 170 for analysis.

Cleaning/Decontamination Modules: Tools dedicated to local decontamination and cleaning tasks. One example is a UV-C light module that can be passed over instrument surfaces or operating surfaces to sterilize them. Another example is a small brush or sponge tool that can apply disinfectant. During turnover (room cleanup) or even intraoperatively if a contamination is detected, the cobot can switch to a cleaning module to sterilize a targeted area or an instrument. This type of end effector helps maintain high sterility standards by autonomously addressing contamination.

Each end effector in the library is designed to be quickly interchangeable via the interface 152, and each may include its own integrated sensors or actuators for enhanced functionality. For instance, a gripper might have fingertip force sensors and an inertial sensor, a suction tool might include a flow rate sensor or pressure sensor to detect occlusions, and a suturing tool might have a small motor to rotate a needle or a sensor to detect needle position. The presence of such sensors/actuators means that when they are connected, the cobot's control system can read those signals and control those actuators, effectively extending the cobot's “hands and eyes” with tool-specific capabilities. This modular approach allows the cobot 100 to perform a broad range of perioperative support tasks by simply switching attachments, rather than requiring multiple dedicated robots. FIG. 1 shows the cobot 100 with one example end effector attached (a gripper 180 in this illustration), and FIG. 2 shows an exploded view of how another tool (e.g., a sensor probe 180) would align and attach to the arm via the interface 152.

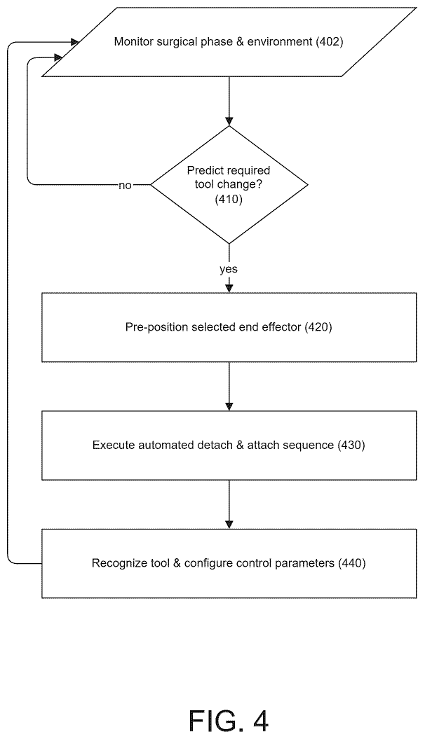

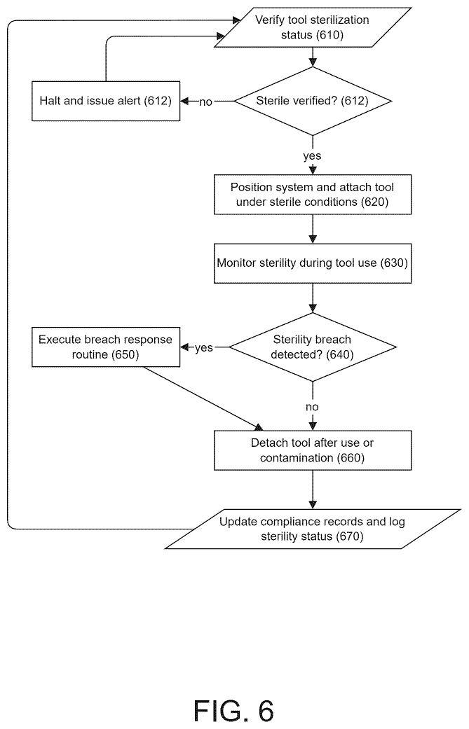

The functional architecture of the cobotic system is shown in FIG. 3, which is a block diagram of the control and recognition systems coordinating the mobile cobot. At the core is the control system 170, which may include one or more processors, microcontrollers, and associated memory running the cobot's operating software. The control system 170 is in communication with all major subsystems of the cobot, including the mobile base 110 drive system, the robotic arm 150 actuators, the end effector interface 152 (and any attached tool), various sensors (cameras, proximity sensors, etc.), and the user interface display 160. FIG. 3 illustrates these connections: for example, control system 170 sends motion commands to the base 110 and arm 150, receives feedback from joint encoders and navigation sensors, interfaces with the recognition module 172 that identifies tools and environment context, and interfaces with the predictive task engine 174 (discussed further with FIG. 4) and the sterility compliance module 176 (discussed with FIG. 6).

One important component shown in FIG. 3 is the End Effector Recognition and Configuration System 172, which automatically detects when an end effector is attached and determines the type or identity of that tool. In one embodiment, each authorized end effector 180 includes an identification tag or chip, for instance, an RFID tag, a one-wire memory device, or a small microcontroller that communicates a unique tool ID upon connection. When the tool is mounted via interface 152, the recognition system 172 reads this ID and cross-references it against a stored database of tool profiles. The control system 170 then configures the cobot's behavior and control parameters according to the attached tool. For example, if the newly attached tool is a suction nozzle, the system will load the control routines for operating suction (e.g., enabling a pump or opening a suction valve) and might limit the arm's speed to account for the tether of suction tubing. If the tool is a delicate sensor probe, the control might reduce applied forces and adjust the arm's posture to protect the sensor. Motion profiles (speed, acceleration limits, safety constraints) are thus dynamically adjusted based on tool type to ensure optimal and safe operation. In some embodiments, the recognition system 172 can also perform a brief self-check or calibration routine specific to the tool, for instance, verifying that a gripper's fingers open/close properly or that a particular sensor is responding, before declaring the tool ready for use.

Importantly, the recognition and control system also implement safeguards to prevent use of incompatible or unauthorized tools. The interface 152 and recognition module 172 may reject any tool that is not recognized or not approved for the current procedure. For example, if someone attempts to attach a tool that has not been sterilized or a tool intended for a different model of cobot, the system will detect an unknown ID and refuse to actuate that tool, possibly displaying a warning on the UI. The control logic can also factor in the surgical context: the cobot may know the planned procedure or current surgical phase (via integration with the hospital's surgical scheduling system or by analyzing the surgeon's actions) and thus ensure that only tools relevant to that context are enabled. In one scenario, if the procedure is, say, a cardiac surgery, the system can lock out any end effector that is not intended for cardiac procedures to avoid confusion or error. Similarly, role-based restrictions can be in place, for instance, certain potentially hazardous tools might require surgeon voice authorization before activation, or only a circulating nurse's console can command a cleaning module to spray disinfectant. These measures guarantee that the cobot 100 operates within safe bounds and uses only the proper instruments at any given time.

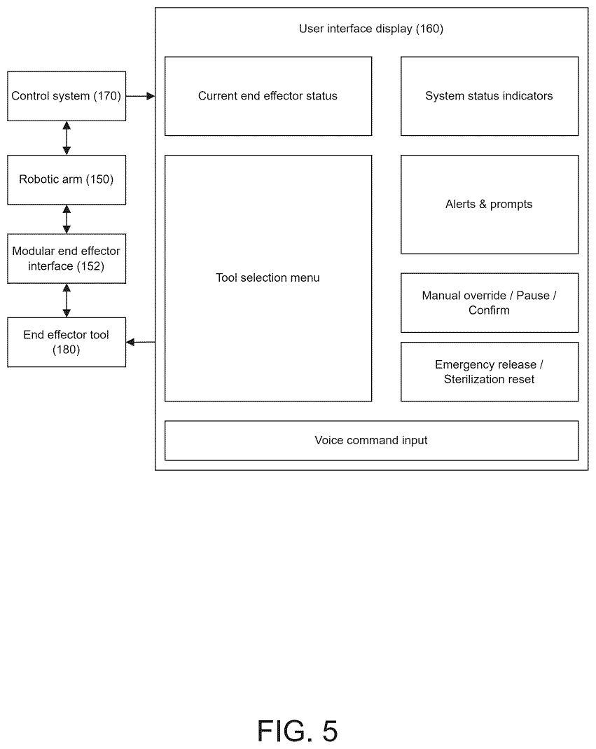

The control system 170 shown in FIG. 3 also encompasses the navigation and safety subsystems for the mobile base. It continuously processes input from cameras and proximity sensors to localize the cobot in the OR and to identify boundaries of the sterile field. The sterile field recognition may be achieved via computer vision (e.g., detecting the draped patient and instrument tables) or by predefined no-go zones set by the user. The control system uses this information to command the base 110 to avoid crossing into sterile areas or colliding with staff. Moreover, FIG. 3′s block diagram includes the communication module 180, which handles wireless data exchange, for example, sending system status or receiving updates from hospital networks. The display 160 (FIG. 5, described later) is also connected, ensuring the user interface always has up-to-date information from the control system. Overall, FIG. 3 shows how the cobot's brain (control system 170) orchestrates the various functional units, recognition, prediction, mobility, manipulation, user interaction, to operate as an integrated perioperative assistant.

To maximize efficiency in the operating room, the cobot 100 employs a Task-Aware End Effector Selection Engine 174, which is depicted conceptually in FIG. 4 as part of a workflow diagram for tool prediction and swapping. The selection engine 174 uses procedural cues, real-time data, and possibly machine learning to anticipate which tool will be needed next, and it prepares the system accordingly. FIG. 4 illustrates an example predictive workflow: the process begins with the cobot monitoring the current surgical phase and environment (step 402). This can include analyzing the live video feed (to recognize what the surgical team is doing or which instrument the surgeon is using), listening for verbal cues (such as a surgeon saying “suction”), and consulting a preloaded surgical procedure model or checklist. For instance, the system might know that after an incision is made, the next typical step is to cauterize or to apply suction to clear blood. Based on such cues, at decision point 410 the engine 174 predicts a required tool change. If a different end effector is likely to be needed soon, the system can either proactively fetch and attach that tool or at least alert the staff of the upcoming suggestion.

When the predictive engine decides to initiate a tool swap, step 420 involves pre-positioning the appropriate end effector. In an autonomous mode, this could mean the cobot 100 drives to a tool staging area or uses an onboard tool storage carousel to pick up the predicted tool in advance. In a semi-autonomous mode, the system might simply highlight the suggested tool on the display (FIG. 5) and wait for a clinician to confirm or trigger the swap. Assuming the tool change is authorized (either automatically by meeting certain criteria or via user confirmation), the cobot executes the automated tool detachment and attachment sequence at step 430. This sequence entails safely pausing any active task, moving the arm 150 to an appropriate position (often away from the patient to avoid contamination), disengaging the current tool (unlocking interface 152), and then securing the new tool onto the interface. The modular coupling allows this sequence to be completed quickly, for example, a latch opens, the arm retracts the old tool and places it in a discard tray or returns it to a holder, then the arm moves to align the new tool and engages the lock. Throughout this process, the selection engine and control system ensure that the base 110 remains outside the sterile field and that no sterile surfaces are breached during the swap (for instance, the arm might move over a safe zone, like a side of the patient that is draped, rather than over the open surgical site, to swap tools). Once the new end effector is attached and recognized (step 440), the control system 170 reconfigures the control parameters for that tool (as discussed earlier) and notifies the surgical team that the cobot is ready with the new instrument. The workflow then continues with the cobot either resuming autonomous assistance or waiting for the next command.

FIG. 4's tool change workflow may interface with external scheduling or preference data as well. In some embodiments, the cobot 100 is connected to the hospital's electronic surgical schedule or a database of surgeon preferences. This allows the predictive engine 174 to know in advance the planned procedure steps and instrument lists. For example, if the schedule indicates a knee arthroscopy, the system can pre-load knowledge that after camera insertion, a shaver tool is often needed. It can thus have the shaver end effector on standby. The engine can also learn over time from past procedures: using machine learning, it might refine its predictions of which tool a particular surgeon typically wants next, thereby becoming more accurate with use. The result is a cobot that not only reacts to requests, but proactively assists by having the right tool at the right time, reducing delays in surgery.

To illustrate the capabilities of the predictive tool selection in concrete scenarios, the following are example surgical support workflows where the cobot's ability to change tools autonomously or semi-autonomously is utilized:

Patient Draping Assistance: At the beginning of a procedure, after the patient is positioned, the cobot 100 anticipates the need for handling sterile drapes. Using its drape holder end effector, the cobot positions itself at the bedside and holds one edge of a large sterile drape (as surgeons typically do). It extends its arm to spread the drape over the patient while surgical staff arrange and secure the drape. The predictive engine knows from the procedure start workflow that draping is required, so it had already attached the sterile drape holder tool when the patient was being positioned. Once draping is complete, the system may autonomously detach the drape holder tool and switch to the next needed tool (for example, a camera or a retractor) without needing to be told. This speeds up the OR setup process.

Autonomous Suction during Bleeding: During a surgical incision or resection, bleeding or fluid buildup can occur unpredictably. The cobot's vision system detects a significant amount of blood in the field (or it hears a clinician call out “I need suction”), and the selection engine immediately selects the suction nozzle end effector. The cobot either automatically swaps out its current tool for the suction tool or prompts the nurse via the UI for permission to do so. Upon attachment, it positions the suction nozzle near the bleeding site and activates suction, removing blood to clear the surgeon's view. This can be done autonomously within seconds of detecting the need, potentially faster than a human could request and receive a suction instrument. If the surgeon then needs a different tool (e.g., an electrocautery instrument to stop the bleed), the cobot can likewise predict and prepare that tool as the next step. The system ensures that fluid suction is performed safely, for instance, monitoring the amount of fluid removed and making sure not to disturb any tissue, and it can either continuously hold the suction in place or hand control back to a human once attached.

Suture Assistance and Tool Handoffs: Near the end of a procedure, the cobot predicts the transition to wound closure. It retrieves a suture assistance tool, such as a needle driver or a suture cutter. If operating in autonomous mode, the cobot may gently take a suture needle (loaded in the needle driver end effector) and position it at the wound edge, ready for the surgeon to take over or to use in stitching under supervision. The cobot could hold one end of a suture thread taut with a gripper while the surgeon ties a knot on the other end, effectively acting as a skilled assistant. The predictive engine knows suturing is imminent because the main surgical tasks (e.g., organ resection) have completed, and it has observed the surgeon reaching for suture material. By having the suture tool already in hand, the cobot can seamlessly step in to assist. If the surgeon at any point prefers to do this manually, they can use the override controls (discussed below) and the cobot will hand off the instrument or stand down. In another embodiment, the cobot performs the suturing task autonomously, defining pressure applied to the suture knot. Once suturing is finished, the cobot can even switch to a cleaning module to disinfect the area or to a sensor probe to scan the closed incision for any residual items, based on predicted post-operative checks.

Instrument Fetching and Transport: Throughout the procedure, the cobot can serve as a runner or instrument table adjunct. For example, if the surgical team needs a particular instrument or implant that is not immediately on the sterile field, the cobot can use its gripper end effector to retrieve the item. Suppose during an orthopedic surgery the surgeon decides to use a different size implant; the cobot's task engine recognizes this request (either from a voice command or an entry on the surgical plan) and navigates to a nearby instrument trolley. Using the gripper 180, it picks up the sterile packaged implant or instrument (possibly from a designated hand-off point where a circulating nurse places it). The mobile base 110 then drives the arm to the surgeon's side to deliver the item. If the item is in a sterile pack, the cobot can present it for a scrub nurse to open. In an alternative workflow, the cobot itself could have a small sterilization chamber or storage compartment and know the inventory of tools it carries, then upon prediction, it simply selects and hands over the requested tool. In either case, by predicting instrument needs, the cobot reduces the wait times for instrument exchanges and reduces the need for a human runner leaving the OR.

These examples demonstrate how the predictive task engine 174 and automated tool changing capability allow the cobot 100 to actively assist with perioperative workflows. FIG. 4's diagram generally encapsulates such scenarios by showing how the system moves from context recognition to tool selection to tool swap and task execution. The result is a more efficient surgery where the cobot anticipates needs and either acts autonomously or provides the tool to the surgical team right when they need it, without a break in the procedure's flow.

An interactive user interface is provided to allow clinicians to monitor the cobot's status and intervene when necessary. FIG. 5 depicts an example user interface display 160 presented on the cobot or a connected device. The display 160 in this embodiment is a touchscreen panel mounted on the cobot's chassis (it could also be a separate tablet or a console screen). This UI shows the available tools and control options to the surgical staff. For instance, a portion of the screen may list icons or names of each end effector in the system's library, highlighting which tool is currently attached to the arm 150 and which tools are on standby. In FIG. 5, the interface 160 includes a graphical indicator of the current end effector (e.g., “Gripper: Attached”), along with a menu of other tools like “Suction”, “Suture Cutter”, “Camera Probe”, etc., that the cobot can swap to. Next to each tool name might be a status indicator (e.g., green if the tool is sterilized and ready, or gray if not available). The interface 160 also displays system status information such as battery level, connectivity, and any alerts (for example, a warning if a tool's sterility is in question or if the cobot's path is blocked).

The user interface provides manual override controls that give human staff final authority over tool changes and other cobot actions. As shown in FIG. 5, a prominent “Manual Override” or “Pause/Confirm” button is available, pressing this would immediately pause any autonomous action and/or require the cobot to seek confirmation before proceeding. In practice, the cobot might flash a notification on screen like “Ready to swap to Suction tool, proceed?” which a nurse or surgeon can accept or decline. The UI also allows staff to manually request a specific end effector at any time, bypassing the autonomous selection. For example, if the surgeon unexpectedly needs the drape holder again, they can tap the “Drape Holder” icon on the display, and the cobot will switch to that tool even if it wasn't predicted. This visual menu of tools with one-touch selection is designed for quick use in the OR environment. Additionally, the interface supports voice-based commands as an alternative or complement. A connected microphone and voice recognition module (part of the control system 170) can interpret simple commands like “Cobot, change to grasper” or “cobot hold camera here,” which the system would then execute, showing the recognized command on the display for confirmation.

FIG. 5 also illustrates that the UI provides real-time feedback on tool status and system decisions. For instance, it may show a timeline or workflow diagram indicating the upcoming predicted tool change. It could say “Next: Prepare Suture Tool” with an option for the user to confirm or adjust that prediction. In case the user disagrees with a prediction, they can manually select a different tool, effectively overriding the Al's choice. This collaborative mode ensures the human team feels in control and can correct the system if needed. The display 160 might further include a log or history window (text or icons) showing recent actions: e.g., “Suction attached at 10:32,” “Suction detached at 10:35,” etc., which aids transparency.

Safety is paramount, so the fail-safe disengagement mechanism is also accessible via the Ul and physical controls. In one embodiment, there is a physical emergency stop button on the cobot 100 that immediately halts all motion and can also trigger the end effector to release whatever it's holding (if safe to do so). Additionally, the UI could have a software “Emergency Release” button that causes the interface 152 to unlock the tool instantly, for example, if a tool malfunctioned or is causing unintentional harm, a staff member can hit this to drop the tool and power it down. A related function is sterilization reset, an option on the UI which the staff can activate if they suspect any contamination (perhaps if someone accidentally touched a non-sterile part of the cobot). Activating the sterilization reset could make the cobot withdraw from the sterile field, mark the currently attached tool as contaminated (prevent further use until re-sterilized), and prompt a cleaning cycle or tool change. As indicated in FIG. 5, the interface 160 would then alert “Sterility breach: tool dropped” or similar, and guide the staff through the recovery steps (like attaching a cleaning module or obtaining a new sterile tool).

The visual layout in FIG. 5 is designed for clarity and ease of use in the hectic OR setting. Large, easy-to-press buttons and clear color-coded signals (green for normal, red for alerts) are used. The UI might integrate with other hospital systems as well; for example, showing a checklist of surgical steps and marking which ones the cobot has assisted with (drape done, instruments delivered, etc.). By giving a real-time window into the cobot's operations and predictions, the interface 160 fosters trust and smooth teamwork between the human surgical team and the robot. In summary, FIG. 5 emphasizes that while the cobot is capable of autonomous decisions, the human operators retain ultimate control through intuitive manual overrides and status visualization. This design aligns with safety guidelines for collaborative robots in surgery, ensuring that a surgeon can always intervene or command the system directly when needed.

FIG. 6 is a flowchart illustrating an example end effector sterilization and compliance protocol executed by the cobot 100 to uphold sterility throughout its operation. Because the cobot frequently changes tools and operates near the sterile surgical field, it implements rigorous checks and procedures at each step of tool handling. The flowchart of FIG. 6 can be described in a series of steps as follows (each step corresponding to a block in the figure):

Initial Sterility Verification: When a tool is selected for use (whether by prediction or user request), the system first verifies the tool's sterilization status (step 610). The cobot's control system 170 checks the database or sensor data for that end effector to ensure it has been properly sterilized and has not exceeded any safe exposure time. In one embodiment, each end effector 180 carries a sterilization indicator tag (for example, an RFID with a field indicating last sterilization time or a chemical indicator strip on the tool that can be optically read). If the verification fails (i.e., the tool is not confirmed sterile or its status is unknown), the system will halt the tool change and issue an alert to the staff (step 612). The UI 160 (FIG. 5) might display “Tool X not sterile, please sterilize or choose another.” This prevents non-sterile equipment from ever entering the sterile field. If the tool is verified sterile, the protocol proceeds.

Tool Attachment under Sterile Conditions: Assuming a sterile tool is available, the cobot positions itself for the swap in a manner that maintains sterility (step 620). The flowchart indicates that the system will move to a safe zone if needed (e.g., over a sterile instrument table or at the periphery of the sterile field) to perform the attachment. The cobot then attaches the tool via interface 152, as described earlier, using only sterile-contact components. During this process, the sterile barrier at the interface ensures that any part of the arm that is not sterile does not touch the tool. Step 620 thus results in the new tool being secured and ready, with the sterile chain unbroken.

Active Monitoring During Use: Once the tool is attached and in use (step 630), the system continuously monitors for any event that could compromise sterility. This includes tracking the position of the end effector relative to the sterile field and non-sterile areas. For example, the cobot's vision system tracks if the tool or arm accidentally moves outside the intended sterile zone (e.g., brushes against a non-sterile surface like the floor or an unprepped area of the patient's body). It also monitors environmental sensors, for instance, if a sterile drape shifts or a non-sterile object comes into contact with the tool. The compliance module 176 in the control system (FIG. 3) uses this data to decide in real time if a sterility breach might have occurred.

Detection of Sterility Breach: If the system detects a potential contamination (decision point 640), it immediately initiates a breach response subroutine (step 650). A breach could be detected by various means: perhaps a sudden drop of the tool (if it slips and touches the floor), a sensor reading indicating biological contamination on the tool's surface, or simply the logic that the tool was moved out of bounds. The response at step 650 is to halt any further use of the potentially contaminated tool and notify personnel. The cobot can freeze its arm in a safe position and flash a warning on display 160 (“Contamination detected: Tool has left sterile field”). At the same time, it may engage an automatic routine to mitigate the breach, for example, if equipped with a cleaning module or UV sterilizer, it could quickly sterilize the tool or area that was contaminated. In one embodiment, the cobot would detach the contaminated tool and place it into a built-in sterilization chamber on the mobile base 110 or onto a return tray for re-sterilization. Alternatively, it might attach a cleaning end effector (like a UV light module) and pass it over the contaminated area (such as a part of the patient's skin or the floor where the tool fell) to neutralize any contaminants. These actions would be done under the guidance or confirmation of the surgical staff as needed. The system also logs the incident (time and nature of breach) as part of compliance records.

Continued Operation or Abortion: If no breach is detected at decision point 640, the tool use continues normally. The system periodically or continuously re-checks sterility (back to step 630) throughout the duration the tool is attached. Once the task is completed and the tool is no longer needed, the cobot will detach the tool (step 660) and either place it aside for post-operative re-sterilization or immediately engage a sterilization cycle if it has that capability. For example, after using a gripper to carry an implant, once done, the cobot might expose the gripper jaws to UV light for a minute to re-sterilize them before the next task. If a breach had occurred and step 650 was executed, the protocol might dictate aborting autonomous operations until human assessment is done. The surgical team can then decide to either replace the cobot's sterile drape, swap in a fresh tool, or in extreme cases, remove the cobot from the sterile field if necessary. FIG. 6 accounts for these possibilities by including branches for handling both normal completion and breach scenarios.

Ensuring Compliance and Logging (step 670): The final part of the flowchart involves updating the sterility status records and ensuring compliance before any subsequent tool change. The system updates its internal log (and the UI display 160) with information such as “Tool X used for 10 minutes, no contamination detected” or “Tool Y marked contaminated, awaiting re-sterilization.” This data is stored in the Data Logging and Analytics Module. The logging system tracks each end effector's usage history and how many sterilization cycles it has undergone, which is important for maintenance and hospital record-keeping. It may alert staff if a tool has exceeded its recommended number of uses or if it's due for preventive maintenance (e.g., sharpening or calibration). Additionally, this module can interface with the hospital's asset management system to update the inventory, for example, flagging that a particular instrument needs to be sent back to central sterile processing after the case. By closing the loop with thorough documentation and compliance checks, the system ensures that each tool change and usage is fully compliant with surgical sterility protocols before moving on.

Through the process outlined in FIG. 6, the cobot 100 maintains a high level of sterility assurance. The combination of sterile barriers, status checks, real-time monitoring, and automated responses to contamination significantly reduces the risk of surgical site infection due to the robot's involvement. Notably, the cobot's awareness of the sterile field means it will, for example, avoid performing a tool change directly over the patient. The control system will instead move the arm to the side or near a known sterile supply table to swap tools, thereby preventing any particles or droplets from a changing tool from falling into the wound. If the robot needs to traverse from one side of the OR to another, it will plan a path around the perimeter of the sterile field, rather than cutting through it. All these behaviors are part of the encoded safety compliance rules.

In summary, the detailed description above, in conjunction with FIGS. 1-6, provides a full enablement of the mobile perioperative cobotic system with modular end effectors. The mobile cobot embodiment offers the flexibility and reach needed in an OR, while the modular end effector interface and rich toolset allow it to perform a wide array of support functions (instrument transport, suction, draping, retracting, drilling, suturing assistance, sensing, cleaning, etc.). The control architecture intelligently recognizes tools, predicts surgical needs, and carries out tool changes either autonomously or under clinician supervision. Throughout its operation, the system upholds sterility via specialized hardware design and strict protocols, and it gives users an intuitive interface for oversight and intervention. Each aspect of the system as claimed, from the mobile base and multi-DOF arm, to the standardized quick-change tool interface, to the predictive selection engine, to the sterility and safety mechanisms, and the user override controls, is described in sufficient detail to demonstrate how the invention can be implemented and used in practice. The foregoing description of embodiments is intended to fully support the claims and enable those skilled in the art to recognize the advantages of this perioperative cobotic system in enhancing surgical workflow and safety.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non-limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

1. A perioperative cobotic system for supporting surgical procedures and related sterile processing tasks, comprising:
a cobotic manipulation platform including a robotic arm attached to a cobotic base, the cobotic base being mobile or stationary and configured for perioperative support tasks, the robotic arm being articulated with multiple degrees of freedom and high-precision control for positioning in constrained or dynamic operating room environments;
a modular end effector interface on the robotic arm, the interface including a standardized mechanical coupling and an electrical connector that allow rapid attachment and detachment of end effector tools, the interface being configured to securely lock an attached tool in place and to support automatic or semi-automatic tool changes without manual intervention while maintaining sterile conditions during the swap;
an interchangeable set of end effector tools comprising a library of task-specific end effectors for perioperative applications, including at least one gripper for instrument transport, at least one suction or irrigation nozzle, at least one sterile drape holder, at least one suture assistance tool, at least one sensor probe, and at least one cleaning or sterilization module for local decontamination, each end effector tool being attachable via the modular interface and wherein one or more of the end effector tools include embedded sensors or actuators to enhance their functionality;
an end effector recognition and configuration system configured to automatically detect a type of end effector tool upon its attachment to the modular interface and to configure control logic of the cobotic system and motion profile according to the detected tool, the recognition system further preventing activation or use of any attached end effector that is incompatible with the current surgical context, task sequence, or user authorization;
a task-aware end effector selection engine operatively coupled to the cobotic system, the selection engine using procedural cues, surgical phase data, or team input to predict and identify required end effector tools in advance, and interfacing with at least one workflow optimization or scheduling system to pre-position or prompt attachment of an appropriate end effector tool ahead of its needed use;
sterility and safety compliance mechanisms including a sterile barrier integrated at the tool interface, an automated sterilization-status check for each end effector tool before use, and control safeguards to prevent deployment of any end effector that has not been verified as sterile, the mechanisms further comprising sensors or monitors that detect proximity of the cobotic system to a sterile field and automatically halt tool changes or movements that could breach sterile field boundaries;
user interaction and override controls comprising a user interface providing at least one of a visual display or voice-command interface for surgical staff to manually request or confirm end effector changes, and a fail-safe override mechanism configured to allow emergency disengagement of an end effector or a sterilization reset of the system; and
a data logging and analytics module configured to track usage history, wear status, and sterilization cycles for each end effector tool and to integrate with a hospital asset management system, thereby providing real-time inventory visibility, supporting predictive maintenance, and ensuring each end effector's readiness for use.

2. The system of claim 1, wherein the cobotic base is a mobile platform equipped with a locomotion drive and navigation sensors for autonomous movement and positioning within the operating room, including the ability to maneuver into appropriate locations without breaching sterile field boundaries.

3. The system of claim 1, wherein the cobotic base is a stationary mounting platform that remains fixed during operation, the robotic arm being of sufficient reach and articulation to service the operative area from the fixed position.

4. The system of claim 1, wherein the modular end effector interface comprises a quick-connect mechanical coupling and a corresponding electrical connector, the mechanical coupling providing secure locking and alignment of the end effector tool to the robotic arm and the electrical connector automatically providing power and data communication to the tool upon attachment.

5. The system of claim 1, wherein the modular end effector interface further comprises an actuated tool changer mechanism configured to automatically detach an end effector tool from the robotic arm and attach a different end effector tool, under control of the system's control logic, thereby enabling hands-free tool swaps while maintaining sterility.

6. The system of claim 1, wherein the end effector recognition system comprises an electronic identification tag associated with each end effector tool and a reader on the cobotic platform, such that when an end effector tool is attached via the interface the reader automatically identifies the tool by reading the identification tag and triggers the corresponding configuration of the control logic.

7. The system of claim 1, wherein the task-aware end effector selection engine is operatively connected to a surgical workflow monitoring system and is configured to receive real-time data about a surgical procedure's progress, so as to automatically select and ready an appropriate next end effector tool based on an upcoming procedural step detected from the surgical phase data.

8. The system of claim 1, wherein the sterility and safety compliance mechanisms include a protective sterile shroud or barrier surrounding the end effector interface to prevent environmental contamination during tool exchanges, and wherein the automated sterilization status check comprises a sensor or indicator that verifies each end effector tool's sterilization before enabling operation of that tool.

9. The system of claim 1, wherein the user interaction interface includes a voice recognition module configured to accept voice commands from operating room staff for requesting an end effector change, and the override controls include a physical emergency-stop or quick-release mechanism on the cobot to immediately disengage an attached end effector in case of a malfunction or safety hazard.

10. The system of claim 1, wherein the data logging and analytics module is further configured to analyze the logged usage and sterilization data for each end effector tool and to generate a maintenance alert or usage report when a predetermined usage threshold or number of sterilization cycles for a particular tool has been reached, thereby prompting inspection or replacement of that tool.

11. The system of claim 1, wherein the user interaction and override controls further comprise a foot-operated control configured to provide an alternative manual override mechanism for the cobotic system's tool-handling operations.

12. A method for facilitating perioperative support tasks using a cobotic system with modular end effectors, the method comprising:
positioning a cobotic base in an operating room at a location proximate to a surgical field, wherein the cobotic base supports a robotic manipulator and a set of interchangeable end effector tools;
anticipating a required end effector tool for an upcoming surgical task by analyzing procedural cues or surgical phase data or by receiving an input from surgical staff or a scheduling system, thereby identifying a specific end effector tool needed imminently;
in response to the anticipation, detaching any end effector tool currently attached to the robotic manipulator and attaching the identified end effector tool to the robotic manipulator via a modular coupling interface, wherein the attachment is performed automatically by the cobotic system without manual intervention;
automatically recognizing the attached end effector tool and configuring the cobotic system's control parameters and motion profile for operation with that tool, including verifying that the attached tool is authorized and meets sterilization requirements before use;
performing a perioperative support action using the robotic manipulator with the attached end effector tool, the action corresponding to the identified surgical task (such as delivering a surgical instrument, suctioning fluid, holding a sterile drape, assisting with suturing, or locally cleaning a surface); and
logging data pertaining to the end effector tool usage and the performed action in a data storage system, including updating the tool's usage history and sterilization status in a hospital asset management or inventory system in real time;
wherein the method further comprises:
pausing movement of the cobotic base or the robotic manipulator during the detaching and attaching step if a proximity sensor indicates that the cobot is too close to a sterile field boundary, and resuming the tool-change operation only when the system is positioned such that the sterile field will not be compromised.

13. The method of claim 12, wherein anticipating the required end effector tool comprises analyzing real-time surgical workflow information to predict the next instrument or support task needed, including detecting a current surgical phase and referencing a predefined procedure plan to determine which end effector tool will be needed next.

14. The method of claim 12, wherein attaching the identified end effector tool comprises actuating an automatic tool changer mechanism on the robotic manipulator to release a first end effector tool and lock the identified end effector tool into place without human touch, thereby maintaining sterile conditions during the tool swap.

15. The method of claim 12, wherein verifying that the attached tool meets sterilization requirements comprises checking a sterilization status indicator associated with the end effector tool and preventing the performing of the support action if the tool is detected as non-sterile or unauthorized.

16. (canceled)

17. The method of claim 12, wherein performing the perioperative support action includes an action selected from: delivering a surgical instrument to the operating field via a gripper end effector, suctioning fluids or smoke via a suction end effector, holding or adjusting a sterile drape via a drape-holding end effector, assisting in suturing by providing or tensioning suture material via a suture tool, or disinfecting a targeted surface via a cleaning end effector module.

18. The method of claim 12, further comprising receiving a manual confirmation or override input from a user prior to attaching the anticipated end effector tool, to ensure surgical team approval and to allow intervention if the selection is not desired, wherein the cobotic system proceeds with the tool attachment only upon receiving the confirmation or proceeds to an emergency stop if an override is triggered.

19. The method of claim 12, further comprising verifying an identity of the attached end effector tool using multiple sensor inputs of different modalities to confirm that the correct tool corresponding to an anticipated requirement is attached.

20. (canceled)

21. An apparatus, comprising:
at least one computer readable storage medium (CRSM) that is not a transitory signal, the at least one CRSM comprising instructions executable by a processor system to:
position a cobotic base in an operating room at a location proximate to a surgical field, wherein the cobotic base supports a robotic manipulator and a set of interchangeable end effector tools; anticipate a required end effector tool for an upcoming surgical task by analyzing procedural cues or surgical phase data or by receiving an input from surgical staff or a scheduling system, thereby identifying a specific end effector tool needed imminently;
in response to the anticipation, detach any end effector tool currently attached to the robotic manipulator and attach the identified end effector tool to the robotic manipulator via a modular coupling interface, wherein the attachment is performed automatically by a cobotic system without manual intervention;
automatically recognize the attached end effector tool and configure the cobotic system's control parameters and motion profile for operation with that tool, including verifying that the attached tool is authorized and meets sterilization requirements before use;
perform a perioperative support action using the robotic manipulator with the attached end effector tool, the action corresponding to the identified surgical task; and
logging data pertaining to the end effector tool usage and the performed action in a data storage system, including updating the tool's usage history and sterilization status in a hospital asset management or inventory system in real time;
wherein the instructions are further executable to:
pause movement of the cobotic base or the robotic manipulator during the detaching and attaching step if a proximity sensor indicates that the cobot is too close to a sterile field boundary, and resuming the tool-change operation when the system is positioned such that the sterile field will not be compromised.
